(19) United States
(12) Patent Application Publication
(10) Pub. No.: US 2023/0140234 A1
(43) Pub. Date: May 4, 2023

(54) DEVICE FOR DELIVERING IONIC MATERIAL AND METHOD OF CONTROLLING THE SAME

(71) Applicants: SAMSUNG ELECTRONICS CO., LTD., Suwon-si (KR); Seoul National University R&DB Foundation, Seoul (KR)

(72) Inventors: JOONSEONG KANG, Suwon-si (KR); Seung-Kyun Kang, Seoul (KR); Sung-Geun Choi, Seongnam-si (KR); YOUNG JUN HONG, Seoul (KR); DON-WOOK LEE, Seoul (KR)

(73) Assignees: SAMSUNG ELECTRONICS CO., LTD., Suwon-si (KR); Seoul National University R&DB Foundation, Seoul (KR)

(21) Appl. No.: 17/731,445

(22) Filed: Apr. 28, 2022

(30) Foreign Application Priority Data
Nov. 4, 2021 (KR) 10-2021-0150719

(51) Int. Cl. A61M 5/165; B01D 61/42; B01D 61/48; B01D 61/52; B01D 61/44

(52) U.S. Cl. CPC A61M 5/165; B01D 61/422; B01D 61/48; B01D 61/52; B01D 61/445; A61M 2005/1657; B01D 2311/2603

(57) ABSTRACT

A device for delivering an ionic material includes a storage module including a reservoir configured to store the ionic material, a bipolar membrane configured to pass the ionic material in a single direction based on an ionic current, electrodes, disposed on a lower end of the reservoir and an upper end of the bipolar membrane, respectively, configured to form an electric field generating the ionic current, and a control module configured to control either one or both of a release amount and a release period of the ionic material passing through the bipolar membrane by adjusting a direction and an intensity of the electric field.

### CROSS-REFERENCE TO RELATED APPLICATIONS

This application claims the benefit under 35 USC § 119(a) of Korean Patent Application No. 10-2021-0150719, filed on Nov. 4, 2021, in the Korean Intellectual Property Office, the entire disclosure of which is incorporated herein by reference for all purposes.

### BACKGROUND

### 1. Field

The following disclosure relates to a device for delivering an ionic material and a method of controlling the device.

### 2. Description of Related Art

To treat patients with a specific disease, for example, such as diabetes or high blood pressure, or deficient in hormones or metabolites such as insulin, a specific drug may be injected or administered to the patients. In this example, the amount of specific drug that needs to be provided to a patient may vary depending on various factors such as the patient's condition and environment. In an example, when an excessive amount of meal than usual is consumed by a diabetic patient, a larger than normal dose of insulin may be provided to the diabetic patient to account for the excessive meal. In another example, when the blood pressure of a hypertensive patient rises above a certain level, an antihypertensive agent may be immediately administered to the hypertensive patient to induce normal blood pressure. In other words, it may be difficult and cumbersome to provide an appropriate amount of drug or medication based on the patient's condition that changes daily.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a device for delivering an ionic material includes a storage module including a reservoir configured to store the ionic material, a bipolar membrane configured to pass the ionic material in a single direction based on an ionic current, electrodes, disposed on a lower end of the reservoir and an upper end of the bipolar membrane, respectively, configured to form an electric field generating the ionic current, and a control module configured to control either one or both of a release amount and a release period of the ionic material passing through the bipolar membrane by adjusting a direction and an intensity of the electric field.

The control module may be further configured to control the ionic material to be released through the bipolar membrane by generating the ionic current based on a forward bias voltage provided by applying a cathode voltage to a region adjacent to a cationic polymer of the bipolar membrane and applying an anode voltage to a region adjacent to an anionic polymer of the bipolar membrane through the electrodes.

The control module may be further configured to control the ionic material not to pass through the bipolar membrane by blocking generation of the ionic current by forming a depletion layer in a bonding portion of the bipolar membrane based on a reverse bias voltage provided by applying an anode voltage to a region adjacent to a cationic polymer of the bipolar membrane and applying a cathode voltage to a region adjacent to an anionic polymer of the bipolar membrane through the electrodes.

The device may further include a sensing module configured to detect either one or both of an amount and a concentration of the ionic material in a human body into which the device is inserted.

The control module may be further configured to determine any one or any combination of any two or more of whether to release the ionic material, the release amount, and the release period of the ionic material according to a detection result of the sensing module, and adjust the direction and the intensity of the electric field based on a result of the determine.

The bipolar membrane may include an anionic polymer having a first permeability to ions having same charges and a cationic polymer having a second permeability to ions having opposite charges, and the second permeability may be greater than the first permeability.

The anionic polymer may include one of sulfonated polyphenylene oxide, sulfonated polyethersulfone, sulfonated polyether ether ketone, sulfonated polystyrene, phosphorylated polyphenylene oxide, phosphorylated polysulfone, and carboxylated polyethylene. The cationic polymer may include one of quaternized polyphenylene oxide, quaternized polysulfone, imidazolated polyphenylene oxide, quaternized polyether ether ketone, amidated polyphenylene oxide, amidated polysulfone, and amidated polyether ether ketone.

The device may further include a drug receptor accommodated in the reservoir. The drug receptor may include the ionic material and any one or any combination of any two or more of phosphate-buffered saline, an aqueous sodium chloride solution, an agarose gel, an alginate gel, and a chitosan gel.

The storage module may be further configured to store a cell generating and secreting a therapeutic factor, and the bipolar membrane may be further configured to store or release the therapeutic factor based on the ionic current.

The storage module may include one of polybutylene adipate terephthalate (PBAT), polyurethane, polyethylene, polysulfone, polydimethylsiloxane, and polymethyl methacrylate.

The electrodes may include any one or any combination of any two or more of platinum, gold, stainless steel, silver, silver chloride, carbon, and oxides thereof.

The device may further include a communication module configured to receive a control signal based on the release amount and the release period of the ionic material.

The device may further include a wireless power reception module configured to wirelessly receive power inducing the electric field.

The device may further include a power supply module configured to supply power inducing the electric field.

In another general aspect, a method of delivering an ionic material in a device includes detecting a concentration of the ionic material in a human body into which the device is inserted, comparing the concentration with a reference concentration, determining any one or any combination of any two or more of whether to release the ionic material and a release time, a release period, a release rate, and a release amount of the ionic material, based on a result of the comparing, and controlling either one or both of a direction and an intensity of an electric field of the ionic material carrier based on the determining.

The determining may include determining to release the ionic material in response to the concentration being less than the reference concentration, and determining any one or any combination of any two or more of the release time, the release rate, and the release amount to release the ionic material in proportion to a difference between the concentration and the reference concentration.

The controlling may include controlling the direction of the electric field to form a forward bias with respect to the ionic material carrier in response to a determination to release the ionic material.

The determining may include determining to block a release of the ionic material in response to the concentration being greater than or equal to the reference concentration.

The controlling may include controlling the direction of the electric field to form a reverse bias with respect to the ionic material carrier in response to a determination to block the release of the ionic material.

A non-transitory computer-readable storage medium may store instructions that, when executed by one or more processors, configure the one or more processors to perform the method above.

Other features and aspects will be apparent from the following detailed description, the drawings, and the claims.

### BRIEF DESCRIPTION OF THE DRAWINGS

FIG. 1 is a block diagram of an example of a device for delivering an ionic material.

FIG. 2 illustrates an example of a structure of an ionic material carrier.

FIGS. 3A and 3B illustrate an example of an operation and a configuration of a device for delivering an ionic material.

FIGS. 4A and 4B illustrate an example of a structure and an operation of a bipolar membrane.

FIG. 5 illustrates an example of a scheme of fabricating a bipolar membrane.

FIG. 6 illustrates an example of a scheme of fabricating an ionic material carrier.

FIG. 7 illustrates an example of bonding a bipolar membrane and a storage module.

FIG. 8 illustrates an example of a graph of a characteristic of a bipolar membrane to which an electric field is applied and an example of a graph of a release amount of an ionic material by a voltage applied to the bipolar membrane.

FIG. 9 illustrates examples of a release amount of an ionic material according to a bias voltage.

FIG. 10 illustrates an example of a change in a release amount of an ionic material according to a thickness of a bipolar membrane for each bias voltage.

FIG. 11 is a block diagram of another example of a device for delivering an ionic material.

FIG. 12 is a block diagram of another example of a device for delivering an ionic material.

FIG. 13 is a flowchart illustrating an example of a method of controlling a device for delivering an ionic material.

FIG. 14 is a flowchart illustrating another example of a method of controlling a device for delivering an ionic material.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

Due to manufacturing techniques and/or tolerances, variations of the shapes shown in the drawings may occur. Thus, the examples described herein are not limited to the specific shapes shown in the drawings, but include changes in shape that occur during manufacturing.

The features of the examples described herein may be combined in various ways as will be apparent after an understanding of the disclosure of this application. Further, although the examples described herein have a variety of configurations, other configurations are possible as will be apparent after an understanding of the disclosure of this application.

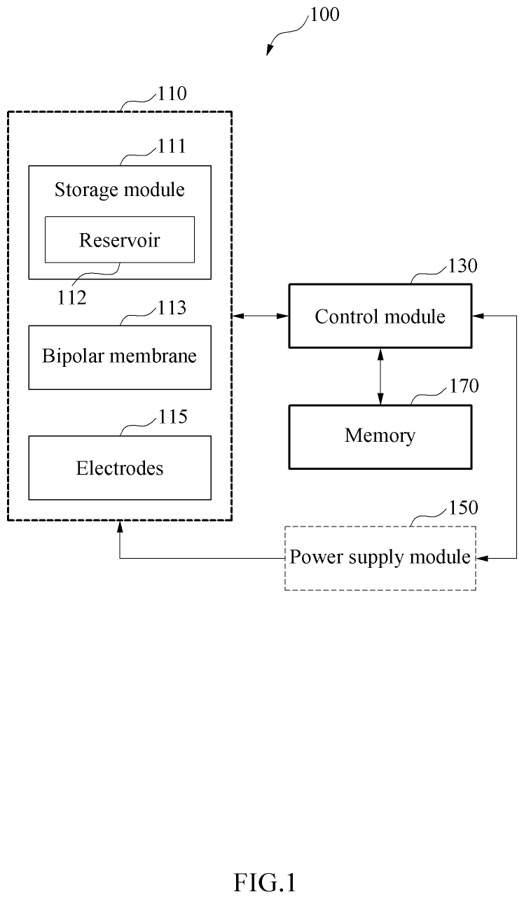

FIG. 1 is a block diagram of an example of a device for delivering an ionic material. Referring to FIG. 1, a device 100 for delivering an ionic material may include a storage module 111, a bipolar membrane 113, electrodes 115, a control module 130, a power supply module 150, and a memory 170. Hereinafter, a device for delivering an ionic material may be referred to as a “delivery device”.

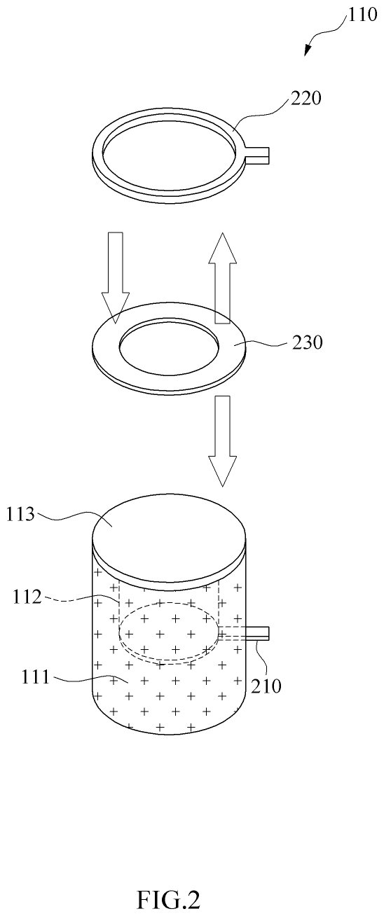

The storage module 111, the bipolar membrane 113, and the electrodes 115 may constitute an “ionic material carrier” 110 configured to deliver or release an ionic material into a body under the control of the control module 130. The ionic material carrier 110 will be described in more detail with reference to FIG. 2 below.

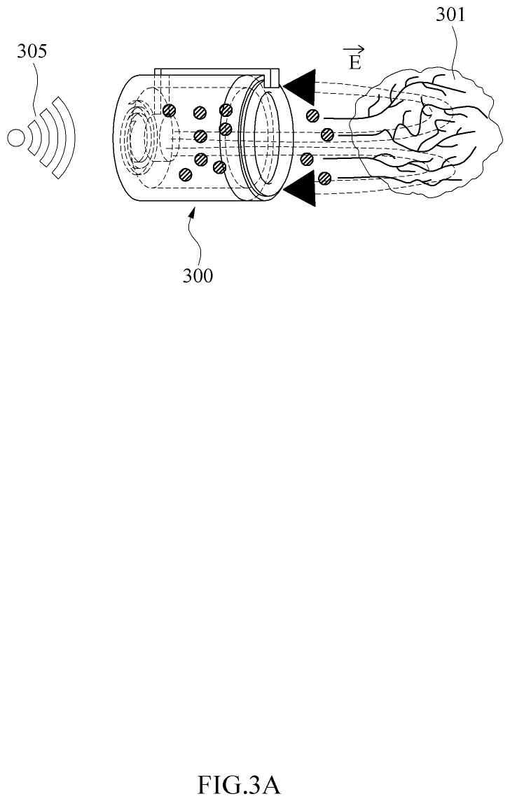

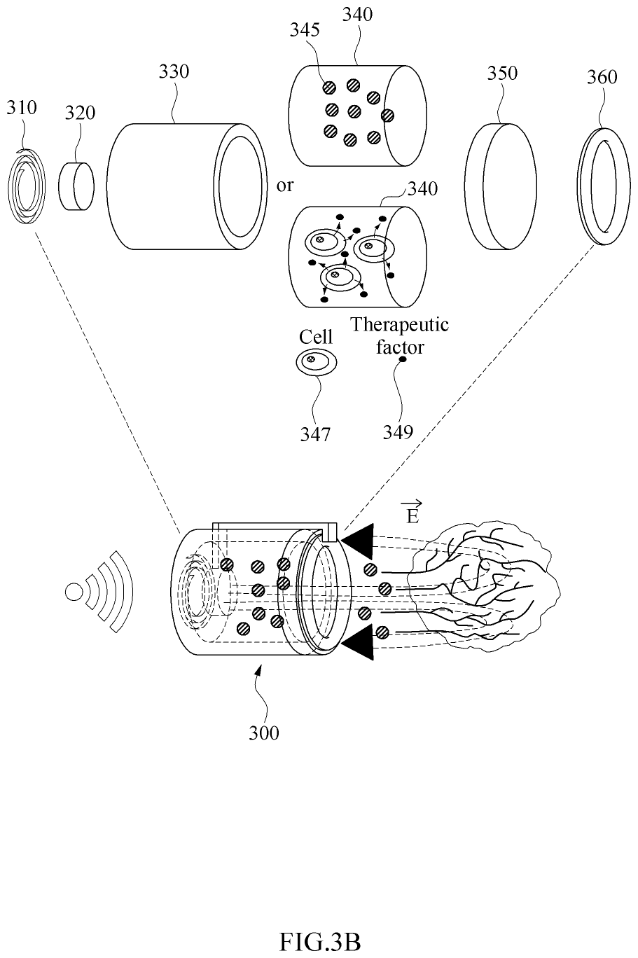

The storage module 111 may store an ionic material to be released into a human body. The storage module 111 may include a reservoir 112 configured to store the ionic material. The ionic material may be included in, for example, a drug receptor (e.g., a drug receptor 340 of FIG. 3B) that will be described below, and may be accommodated in the reservoir 112. The ionic material may include, for example, a neurotransmitter, a therapeutic factor generated and/or secreted by cells, and the like, in addition to the drug for treating a diseased area. Depending on examples, the ionic material may include a material for heavy metal removal, a material for wastewater treatment, a material for desalination, and the like, but is not limited thereto. The term “ionic material” used herein may be construed to include all various materials and factors that may be ionized and moved by selective permeation through the bipolar membrane 113 and/or that may be moved by an electric field provided to the bipolar membrane 113. The ionic material may include, for example, a material that is processed to exhibit polarity because charged nanoparticles are attached to the material, but is not limited thereto.

The drug receptor, a solvent material (e.g., liquid or gel) with high solubility of an ionic material, may enable a high-concentration ionic material to be stored in an ionized state in the storage module 111. Here, since ions other than the drug are present in the solvent material, an ionic current, that is, an ion conductive current may flow through the bipolar membrane 113 if an electric field is applied. The ionic material being “included” and accommodated in the drug receptor may indicate that the ionic material is stored in the reservoir 112 in a state of being dissolved in the drug receptor in a liquid (solvent) or gel state.

The bipolar membrane 113 may allow the ionic material stored in the reservoir 112 to pass therethrough in one direction by an ionic current. Allowing the ionic material to “pass through” the bipolar membrane 113 may be construed to include selective permeation of the ionic material. Here, the “one direction” may correspond to, for example, one direction from the reservoir 112 to the outside of the bipolar membrane 113.

The bipolar membrane 113 may selectively release the ionic material stored in the reservoir 112 based on the presence or absence of the ionic current. The bipolar membrane 113 may seal the reservoir 112 to prevent the stored ionic material from flowing out of the reservoir 112. “Sealing” of the reservoir 112 may merely indicate blocking content (e.g., an ionic material) stored in the reservoir 112 from flowing out of the reservoir 112 if an ionic current is not generated, and may be construed to indicate that a release of an ionic material by selective permeation of the ionic material by an ionic current and/or permeation of components with a small particle size such as water in body fluids through the bipolar membrane 113 are not blocked.

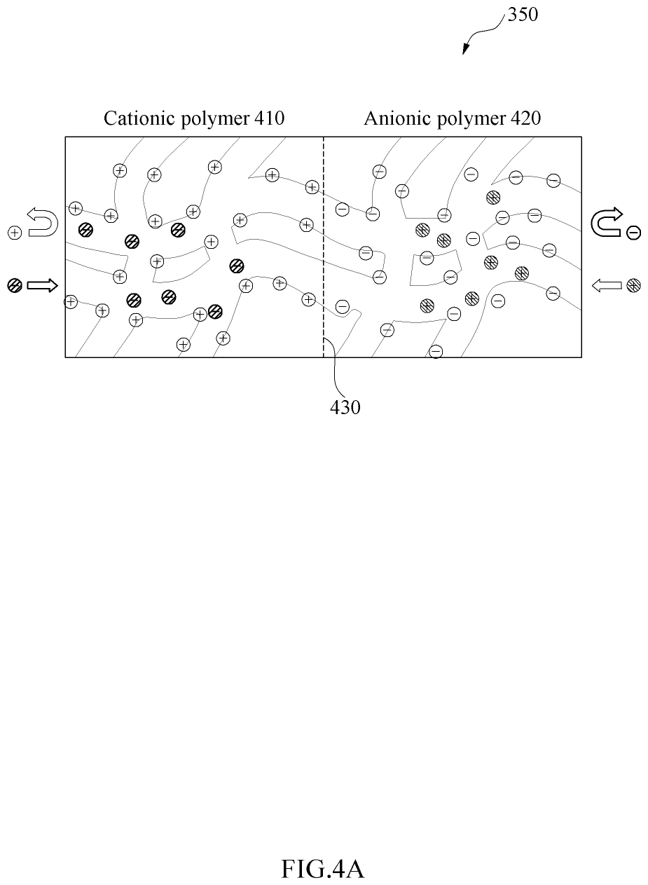

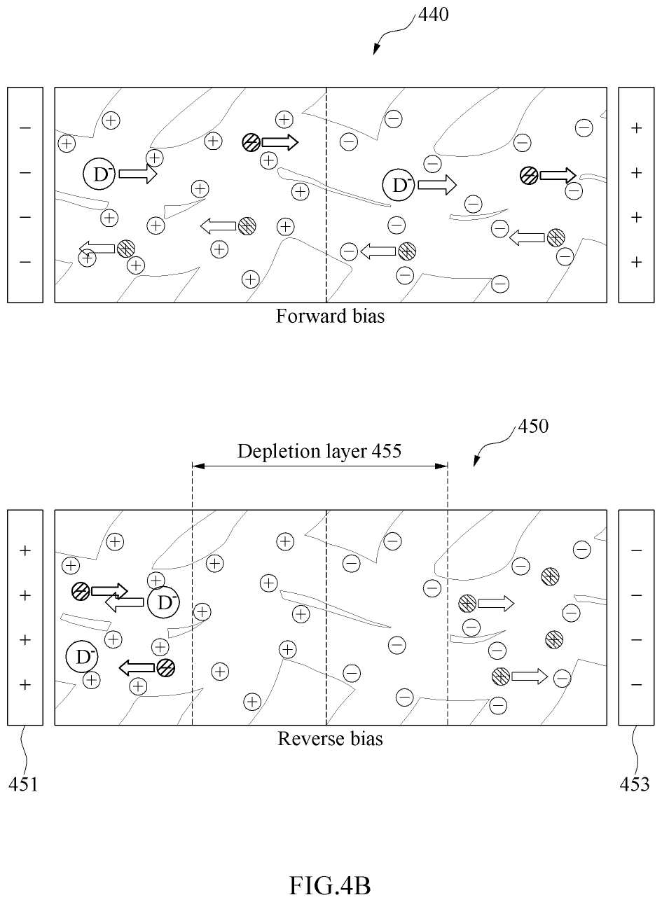

The bipolar membrane 113 may be, for example, a membrane with both poles formed by bonding a cationic polymer and an anionic polymer, as shown in FIG. 4A below.

For example, the bipolar membrane 113 may be formed by bonding a cationic polymer and an anionic polymer, which have a first permeability to ions having the same charges and a second permeability to ions having opposite charges greater than the first permeability. The bipolar membrane 113 may perform a function of an ionic diode that determines whether to allow the ionic current to flow or block the ionic current according to a direction of an electric field generated around the bipolar membrane 113. A structure and operation principle of the bipolar membrane 113 will be described in more detail with reference to FIGS. 4A and 4B below.

Depending on examples, the storage module 111 may further store a cell that generates and/or secretes a therapeutic factor. In this example, the bipolar membrane 113 may store and/or release the therapeutic factor by an ionic current. An example in which the storage module 111 stores cells will be described in more detail with reference to FIG. 3B below.

The electrodes 115 may function to convert electrical energy into an ionic current. The electrodes 115 may be provided in, for example, a lower end of the reservoir 112 and an upper end of the bipolar membrane 113, as shown in FIG. 3B below, and may form an electric field that generates an ionic current if electrical energy is applied.

The control module 130 may control either one or both of a release amount and a release period of the ionic material passing through the bipolar membrane 113 by adjusting a direction in which the electric field is formed and the intensity of the electric field through voltage control.

The control module 130 may control the ionic material to be released through the bipolar membrane 113, by generating an ionic current by a forward bias voltage. The forward bias voltage may be provided by applying a cathode voltage to a region adjacent to a cationic polymer of the bipolar membrane 113 and applying an anode voltage to a region adjacent to an anionic polymer of the bipolar membrane 113 through the electrodes 115.

In addition, the control module 130 may control the ionic material not to be released through the bipolar membrane 113 by blocking the generation of an ionic current by forming a depletion layer (e.g., a depletion layer 455 of FIG. 4B) in a bonding portion (e.g., a bonding portion 430 of FIG. 4A) of the bipolar membrane 113 by a reverse bias voltage. The reverse bias voltage may be provided by applying an anode voltage to a region adjacent to the cationic polymer of the bipolar membrane 113 and applying a cathode voltage to a region adjacent to the anionic polymer of the bipolar membrane 113 through the electrodes 115. The control module 130 may be in the form of a processor or a wireless circuit but is not limited thereto.

The power supply module 150 may supply power for inducing an electric field to the delivery device 100 when the delivery device 100 is inserted into a human body. The power supply module 150 may supply power to, for example, the ionic material carrier 110, the control module 130, and the memory 170. The power supply module 150 may be, for example, one of a battery and a power generation device that supplies power to the delivery device 100 by itself. The battery may be, for example, a rechargeable battery that may be recharged, or a non-rechargeable battery, but is not limited thereto.

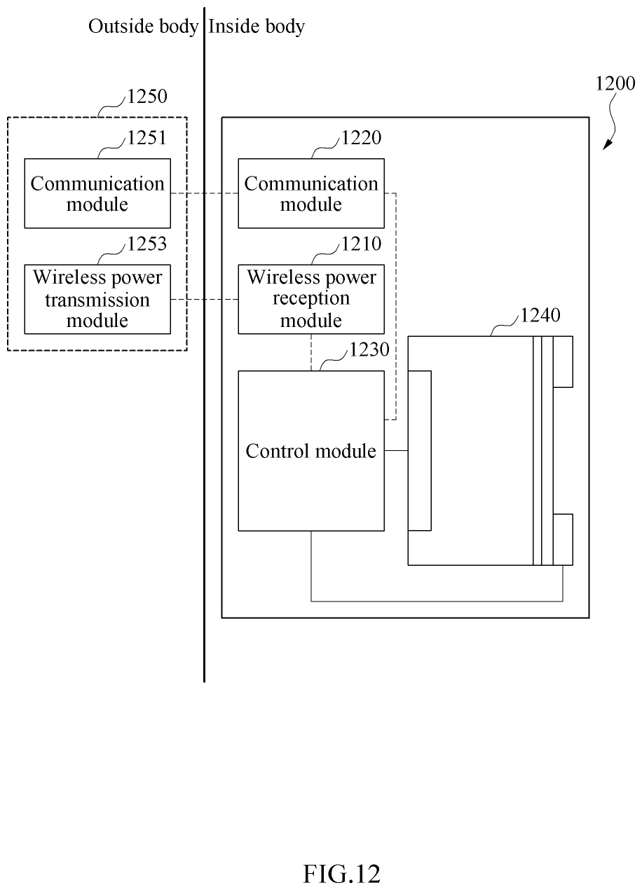

For example, the power supply module 150 may further include a wireless power reception module (e.g., a wireless power reception module 1210 of FIG. 12) configured to wirelessly receive power from the outside of the delivery device 100 and induce an electric field. In this example, the delivery device 100 may charge a rechargeable battery by power wirelessly supplied through the wireless power reception module, or may provide a bias voltage to the ionic material carrier 110.

The memory 170 may store executable instructions to be executed in the control module 130. In addition, the memory 170 may store a variety of information generated in a processing process of the control module 130. Also, the memory 170 may store a variety of data and programs. The memory 170 may include, for example, a volatile memory or a non-volatile memory.

The control module 130 may execute executable instructions included in the memory 170. When the instructions are executed by the control module 130, the control module 130 may perform an operation such as controlling a release of the ionic material from the ionic material carrier 110.

The control module 130 may execute a program and control the delivery device 100. Codes of the program executed by the control module 130 may be stored in the memory 170.

In addition, the delivery device 100 may perform at least one method that will be described below with reference to FIGS. 1 to 14, or a scheme corresponding to the at least one method. The delivery device 100 may be a hardware-implemented electronic device with a physically structured circuit to execute desired operations. For example, the desired operations may include codes or instructions included in a program. The hardware-implemented delivery device may include, for example, a microprocessor, a central processing unit (CPU), a graphics processing unit (GPU), a processor core, a multi-core processor, a multiprocessor, an application-specific integrated circuit (ASIC), a field-programmable gate array (FPGA), and a neural processing unit (NPU).

Since an ionic material (e.g., drug) is released to a portion in direct contact with the ionic material carrier 110 in the delivery device 100, an effect of the drug may be focused on a diseased area, and an exposure of a non-diseased area to the drug may be minimized. Thus, side effects of the drug may be reduced, and effects of the drug may be maximized.

In addition, since the delivery device 100 operates based on a movement of the ionic material by electrophoresis, drug delivery may not be affected by a direction of gravity. Thus, a degree of freedom of a direction and/or a location in which the delivery device 100 is inserted into a human body may be enhanced.

The delivery device 100 may be, for example, an implantable drug delivery device for treatment of a diseased area, a therapeutic/cosmetic drug delivery device in the form of a patch attached to skin, or an implantable neural signal transmitting device (e.g., a cochlear implant) that operates based on a neurotransmitter. In addition, the delivery device 100 may be used in, for example, an energy harvesting device, an energy storage device, a heavy metal removal device, a wastewater treatment device, a desalination device, and a dialysis device which are based on selective permeation properties of ionic materials and/or ion migration properties using an electric field.

FIG. 2 illustrates an example of a structure of an ionic material carrier. FIG. 2 illustrates an arrangement structure of components of the ionic material carrier 110.

Electrodes of the ionic material carrier 110 may function to change electrical energy into an ionic current, and may include a first electrode 210 disposed in the storage module 111 and a second electrode 220 disposed outside the storage module 111. The first electrode 210 and the second electrode 220 may correspond to, for example, the electrodes 115 of FIG. 1.

For example, the first electrode 210 may be disposed in a lower end of the reservoir 112 in which the ionic material is stored in the storage module 111, and the second electrode 220 may be disposed in an upper end of the bipolar membrane 113. As electrical energy is applied to the first electrode 210 and the second electrode 220, an electric field for generating an ionic current may be formed in the bipolar membrane 113.

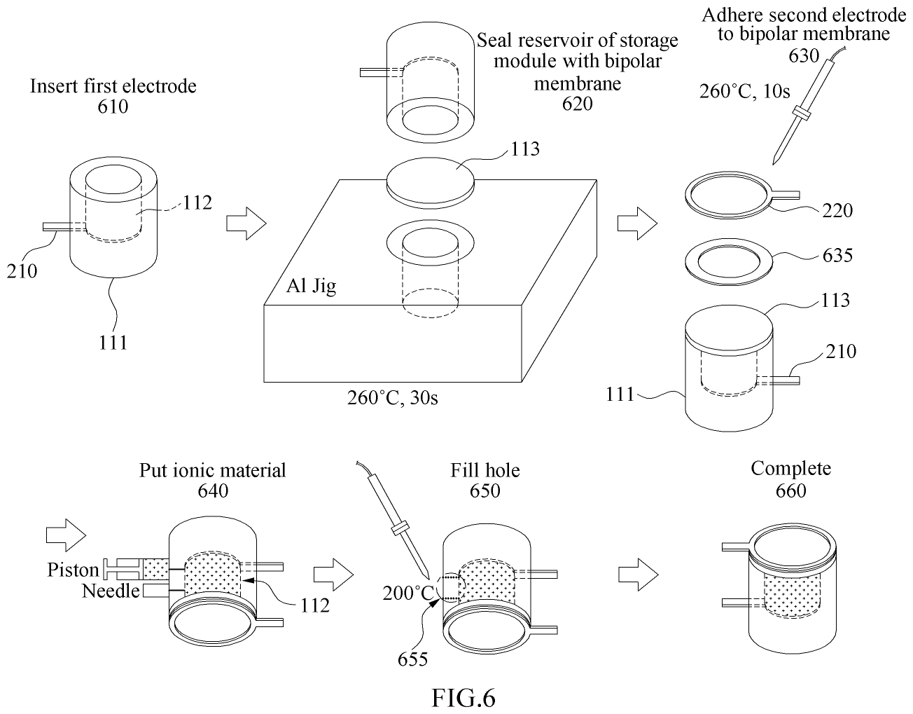

The bipolar membrane 113 may be disposed on the storage module 111. An adhesive film 230 formed of a thermoplastic material in a ring shape may be disposed between the bipolar membrane 113 and the second electrode 220 to help adhesion of the bipolar membrane 113 and the second electrode 220, which will be described in more detail with reference to FIG. 6.

FIGS. 3A and 3B illustrate an example of an operation and a configuration of a delivery device.

FIG. 3A illustrates an example of an operation of a delivery device 300. The delivery device 300 may wirelessly receive power 305 from the outside, and induce an electric field for an ionic material carrier by the power 305 to release an ionic material to an affected area 301 in a human body.

In an example, the delivery device 300 may release the ionic material by an ionic current generated in response to a forward bias voltage being applied to a bipolar membrane (e.g., a bipolar membrane 350) through electrodes (e.g., a first electrode 320 and a second electrode 360). In another example, the delivery device 300 may block a release of the ionic material by a depletion layer formed in a bonding portion of the bipolar membrane 350 in response to a reverse bias voltage being applied to the bipolar membrane 350 through the first electrode 320 and the second electrode 360.

FIG. 3B illustrates, in detail, a configuration of the delivery device 300. For example, the delivery device 300 may include a wireless circuit 310, the first electrode 320, a storage module 330, the drug receptor 340, the bipolar membrane 350, and the second electrode 360.

The wireless circuit 310 may wirelessly receive power from the outside of the delivery device 300 to supply power for the operation of the delivery device 300. The wireless circuit 310 may include, for example, a passive element, such as a resistor, a capacitor, or an inductor, an active element, such as a diode or a transistor, and/or an electronic element chip, such as a microcomputer, an electrostatic circuit, or a regulator. In the wireless circuit 310, a conductive wire for connecting respective elements may include, for example, a metal material such as gold (Au) or platinum (Pt) with high conductivity. A dielectric material of a capacitor may include, for example, silicon dioxide (SiO₂), silicon nitride (Si₃N₄), or magnesium oxide (MgO), which is a metal oxide, having a low conductivity. The wireless circuit 310 may correspond to, for example, the control module 130 described above with reference to FIG. 1.

Electrodes (e.g., the first electrode 320 and the second electrode 360) may function to change electrical energy to ionic current. The first electrode 320 and the second electrode 360 may include, but are not limited to, for example, any one or any combination of platinum (Pt), gold (Au), stainless steel (SUS), silver (Ag), silver chloride (AgCl), carbon (C), and oxides thereof.

The first electrode 320 may be disposed in the storage module 330, for example, in a lower end of a reservoir included in the storage module 330. The first electrode 320 may have, for example, a disc shape or a mesh shape, but is not limited thereto.

The storage module 330 may function to store the ionic material to be released into a human body. For example, the storage module 330 may include a polymer such as polybutylene adipate terephthalate (PBAT), polyurethane, polyethylene, polysulfone, polydimethylsiloxane, or polymethyl methacrylate.

The drug receptor 340, a solvent material with a high solubility of an ionic material (e.g., drug), may enable a drug with a high concentration to be stored in an ionized state in the storage module 330. The drug receptor 340 may allow an ion conductive current to flow in the delivery device 300 when an electric field is applied, due to ions other than the drug in a solvent. The drug receptor 340 may be accommodated in the reservoir of the storage module 330. The drug receptor 340 may include, but is not limited to, for example, phosphate-buffered saline, an aqueous sodium chloride solution, an agarose gel, an alginate gel, and a chitosan gel. The drug receptor 340 may have various states, for example, a gel phase, a liquid phase, a solid phase, and the like.

The bipolar membrane 350 may be fabricated by attaching a cationic polymer and an anionic polymer, as shown in FIG. 4A below. A scheme of attaching the cationic polymer and the anionic polymer to fabricate the bipolar membrane 350 may include, for example, an attachment scheme by thermocompression, an attachment scheme using a solvent, and an attachment scheme through spin coating, but is not limited thereto. The bipolar membrane 350 may correspond to, for example, the bipolar membrane 113 of FIG. 1.

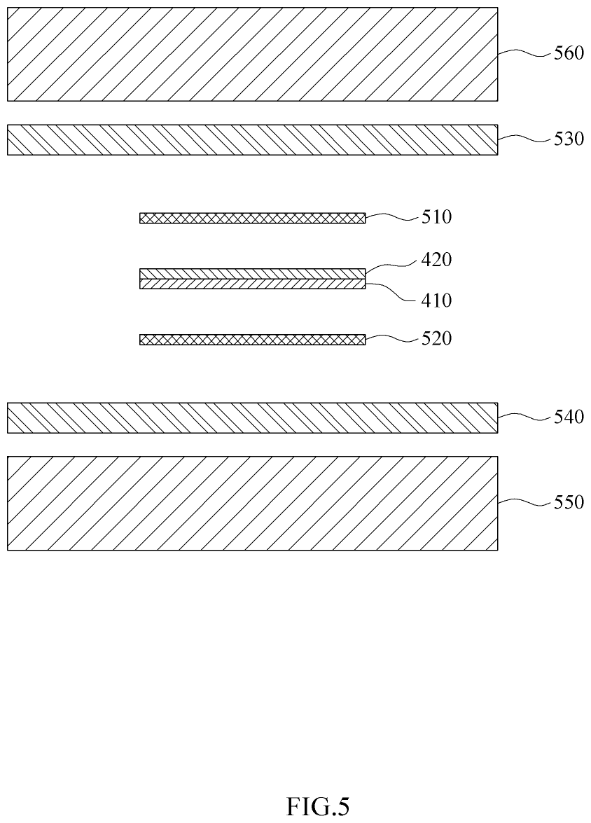

Each of ionic polymers on both sides of the bipolar membrane 350 may have a low permeability to ions having the same charges as its own charges and may have a high permeability to ions having opposite charges. An example of a structure and an operation of the bipolar membrane 350 will be described below with reference to FIGS. 4A and 4B, and a scheme of fabricating the bipolar membrane 350 will be described in more detail with reference to FIG. 5 below.

The second electrode 360 may be disposed outside the storage module 330, for example, in an upper end of the bipolar membrane 350. The second electrode 360 may have, for example, a ring shape or a mesh shape, but is not limited thereto.

In an example, the delivery device 300 may also store a cell 347 that secretes a therapeutic factor 349 in the storage module 330, instead of storing drug 345.

The delivery device 300 may accumulate the therapeutic factor 349, continuously generated from the cell 347, in the storage module 330. The delivery device 300 may release therapeutic factors into a body by an amount desired according to a patient's situation by selective permeation of the therapeutic factor 349 and/or movement of the therapeutic factor 349 using an electric field. The delivery device 300 may use cells 347 that generate therapeutic factors 349, thereby supplying the therapeutic factors 349 for a relatively long period with a minimal surgical operation.

The cell 347 may be, for example, a mesenchymal stem cell (MSC) or a cell that induces directional migration of white blood cells, but is not limited thereto. In addition, the therapeutic factor 349 generated by the cell 347 may be, for example, microvesicles derived from stem cells, cytokines which are one of immune proteins contained in blood, chemokine secreted by cells that induce directional migration of white blood cells, a growth factor, a microRNA (miRNA), or a messenger RNA (mRNA), but is not limited thereto. The therapeutic factor 349 may help a damaged organ or tissue recover itself.

The delivery device 300 may continue to supply dissolved oxygen and nutrients used for a growth of the cell 347 due to the porosity of the bipolar membrane 350, and accordingly, the cell 347 transplanted into the storage module 330 may be preserved in the storage module 330 instead of being lost. In addition, an inflow of a host immune cell incapable of passing through the bipolar membrane 350 may be prevented, and thus an immune response of a human body caused by a cell transplantation may be inhibited.

FIGS. 4A and 4B illustrate an example of a structure and an operation of a bipolar membrane. FIG. 4A illustrates a bipolar membrane 350 in which a cationic polymer 410 and an anionic polymer 420 are bonded.

Each ionic polymer on both sides of the bipolar membrane 350 may have a low permeability to ions having the same charges as its own charges and may have a high permeability to mobile ions having opposite charges.

For example, the cationic polymer 410 may have a low permeability to cations and a high permeability to anions. The anionic polymer 420 may have a high permeability to cations and a low permeability to anions.

The cationic polymer 410 may include, for example, one of quaternized polyphenylene oxide, quaternized polysulfone, imidazolized polyphenylene oxide, imidazole-substituted polyaniline ether ketone, quaternized polyether ether ketone, amidated polyphenylene oxide, amidated polysulfone, and amidated polyether ether ketone, but is not limited thereto.

The anionic polymer 420 may include, for example, one of sulfonated polyphenylene oxide, sulfonated polysulfone, sulfonated polyethersulfone, sulfonated polyether ether ketone, sulfonated polystyrene, phosphorylated polyphenylene oxide, phosphorylated polysulfone, carboxylated polyethylene, and polyacrylic acid, but is not limited thereto.

FIG. 4B illustrates examples of the electrical behavior of the bipolar membrane 350 to release an ionic material. An example 440 may be an electrical behavior of the bipolar membrane 350 in a forward bias, and an example 450 may be an electrical behavior of the bipolar membrane 350 in a reverse bias.

Due to selective ionic permeability of opposite phase characteristics of both ends of the bipolar membrane 350, the bipolar membrane 350 may have properties of an ionic diode that generates an ionic current based on a direction of an electric field applied to the bipolar membrane 350.

For example, in the example 440, a forward bias voltage may be provided by applying a cathode voltage to a region adjacent to the cationic polymer 410 and by applying an anode voltage to a region adjacent to the anionic polymer 420. In this example, an anion that moves close to the bonding portion 430 in the cationic polymer 410 may pass through the anionic polymer 420 by an electric force. A cation that moves close to the bonding portion 430 in the anionic polymer 420 may pass through the cationic polymer 410 by an electric force. Accordingly, an ionic current may flow in the bipolar membrane 350.

In the example 450, a reverse bias voltage may be provided by applying an anode voltage to a region adjacent to the cationic polymer 410 and applying a cathode voltage to a region adjacent to the anionic polymer 420. In this example, an anion in the cationic polymer 410 may move in a direction of the positive voltage electrode 451, and a cation in the anionic polymer 420 may move in a direction of a negative voltage electrode 453, so that the depletion layer 455 with a relatively small number of ions may be formed in the bonding portion 430 between the cationic polymer 410 and the anionic polymer 420. The ionic current may be blocked when the depletion layer 455 is formed in the bipolar membrane 350. Accordingly, the release of the ionic material through the bipolar membrane 350 may also be blocked.

FIG. 5 illustrates an example of a scheme of fabricating a bipolar membrane. FIG. 5 illustrates a process of fabricating a bipolar membrane (e.g., the bipolar membrane 113 of FIG. 1 and the bipolar membrane 350 of FIG. 3B) by a thermocompression scheme using thermocompressors 550 and 560.

To fabricate a bipolar membrane, polymer films 510 and 520 and metal plates 530 and 540 may be sequentially disposed on both ends of the cationic polymer 410 and the anionic polymer 420 that are bonded. Subsequently, top and bottom of sequentially arranged materials may be thermally compressed by thermocompressors 550 and 560, to fabricate the bipolar membrane. For example, a pressure of 5 kgf/cm², a temperature of 200° C., and 1 minute may be provided as thermocompression conditions of the thermocompressors 550 and 560. Each of the metal plates 530 and 540 to provide the pressure of 5 kgf/cm² may weigh, for example, 5 kg. It can be confirmed through experiments that if the bipolar membrane is fabricated by thermocompression at 200° C., the bipolar membrane has a constant ionic current characteristic and that an on-off ratio of a current in a forward bias state to a current in a reverse bias state is maximized.

FIG. 6 illustrates an example of a scheme of fabricating an ionic material carrier. FIG. 6 illustrates processes 610 to 660 to fabricate an ionic material carrier.

In process 610, the first electrode 210 may be inserted into the reservoir 112 of the storage module 111. For example, the first electrode 210 may be attached to the storage module 111 by inserting the first electrode 210 into the lower end of the reservoir 112 in the storage module 111 and applying heat.

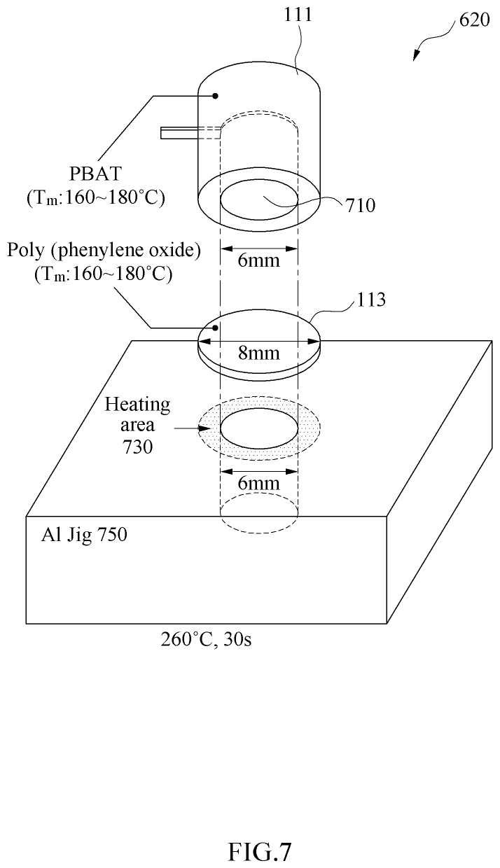

In process 620, the reservoir 112 of the storage module 111 may be sealed with the bipolar membrane 113. In this example, sealing may be performed using, for example, a metal jig heated to a temperature of 200 to 300° C. The metal jig may be, for example, an aluminum (Al) jig. In process 620, for example, the reservoir 112 of the storage module 111 and an annular edge portion of the bipolar membrane 113 other than a passage through which drug moves may be melted and thermally attached by heating the metal jig to 260° C., which is a melting temperature of polyphenylene oxide, for 30 seconds. A scheme of bonding the reservoir 112 of the storage module 111 and the bipolar membrane 113 will be described in more detail with reference to FIG. 7 below.

In process 630, the second electrode 220 may adhere to a top surface of the bipolar membrane 113. The bipolar membrane 113 and the second electrode 220 may be adhered by, for example, melting an adhesive film 635 disposed between the bipolar membrane 113 and the second electrode 220. The adhesive film 635 may be formed of a thermoplastic material in a ring shape. For example, the bipolar membrane 113 and the second electrode 220 may be attached by melting the adhesive film 635 by heating the second electrode 220 to a temperature of 200 to 300° C. for 10 seconds with a hot iron.

In process 640, for example, an ionic material (e.g., drug) may be put into the reservoir 112 of the storage module 111 using a needle and/or a piston. The ionic material may be included in any one or any combination of phosphate-buffered saline, an aqueous sodium chloride solution, an agarose gel, an alginate gel, and a chitosan gel, and may be put into the reservoir 112.

In process 650, a hole 655 of the storage module 111 drilled by a needle when drug is injected in process 640 may be filled by melting an edge portion of the hole 655 using a hot iron, to complete fabricating of the ionic material carrier in process 660.

Subsequently, by electrically connecting a wireless circuit and one side of the first electrode 210 in the ionic material carrier, the wireless circuit may control the operation of the ionic material carrier.

FIG. 7 illustrates an example of bonding a bipolar membrane and a storage module. FIG. 7 illustrates the storage module 111 and the bipolar membrane 113 bonded through local heating based on an aluminum (Al) jig 750.

For example, it may be assumed that the storage module 111 is formed of PBAT with a melting temperature (Tm) of 160 to 180° C., and the bipolar membrane 113 is formed of polyphenylene oxide with a melting temperature (Tm) 177 to 222° C. In addition, it may be assumed that an upper end 710 of a reservoir of the storage module 111 has a diameter of 6 mm, and the bipolar membrane 113 has a diameter of 8 mm.

To seal the upper end 710 of the reservoir by the bipolar membrane 113, a heating area 730 of the Al jig 750 may be heated to 260° C. for 30 seconds such that the bipolar membrane 113 may be sufficiently melted. In this example, the size of the heating area 730 may be greater than or equal to 6 mm that is the diameter of the upper end 710 of the reservoir, and may be less than or equal to 8 mm that is the diameter of the bipolar membrane 113.

By the heating area 730 heated to 260° C., the upper end 710 of the reservoir of the storage module 111 and the annular edge portion of the bipolar membrane 113 other than the passage through which drug moves may be thermally attached and bonded.

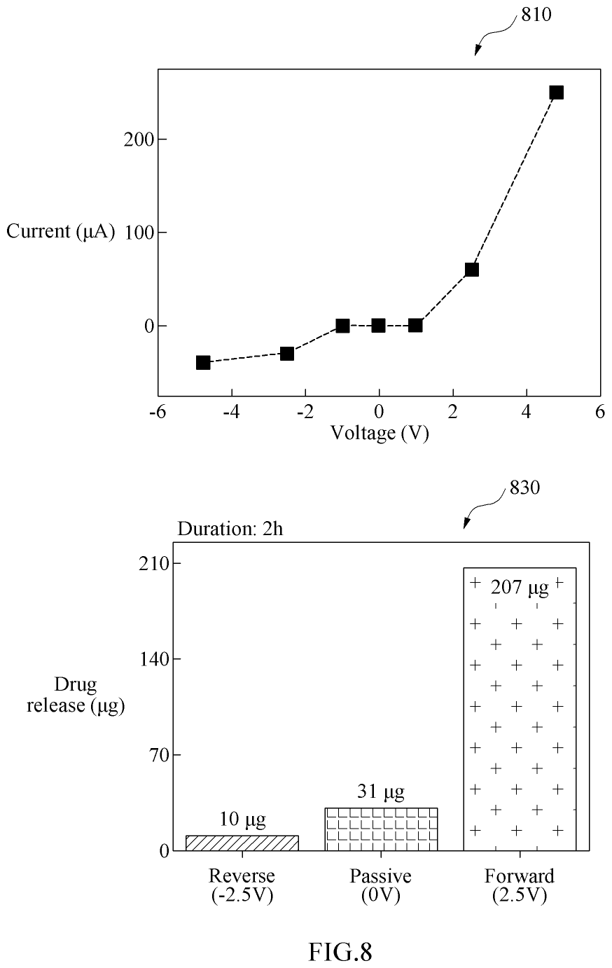

FIG. 8 illustrates an example of a graph of a characteristic of a bipolar membrane to which an electric field is applied and an example of a graph of a release amount of an ionic material by a voltage applied to the bipolar membrane.

Referring to FIG. 8, a graph 810 may represent a characteristic of an ionic diode by a bipolar membrane to which an electric field is applied. As shown in the graph 810, it can be confirmed that as an intensity of the electric field applied to the bipolar membrane is greatly adjusted, an amount of an ionic current generated also increases.

A graph 830 may represent an amount (e.g., a drug release(pg)) of an ionic material (e.g., drug) to be released from the bipolar membrane when a voltage applied to the bipolar membrane includes, for example, a reverse bias voltage, a passive voltage, and a forward bias voltage.

In an example, a delivery device may form an electric field in the bipolar membrane to control a drug release amount based on the bipolar membrane. The graph 830 may show an amount of an ionic current and a drug release amount according to a voltage applied to a periphery of the bipolar membrane. For example, the amount of ionic current may be obtained by measuring a flowing current by connecting a potentiostat-galvanostat between an ionic material carrier and a control module or a wireless circuit. The drug release amount may be confirmed by collecting an electrolyte after the drug is released from the ionic material carrier submerged in the electrolyte and measuring the drug concentration in the electrolyte.

In the graph 830, a “forward bias state” in which a cathode voltage is applied to a first electrode of the delivery device and an anode voltage is applied to a second electrode may be defined as a positive voltage, and a “reverse bias state” in which an anode voltage is applied to the first electrode of the delivery device and a cathode voltage is applied to the second electrode may be defined as a negative voltage.

In the graph 830, it can be seen that an intensity of current measured when the negative voltage is applied (in the reverse bias state) is less than an intensity of current measured when the positive voltage is applied (in the forward bias state). In addition, the characteristic of the ionic diode may be determined based on a difference in ion conductivity according to a polarity of a voltage. It can be found based on the graph 830 that the release amount of the ionic material increases to 207 pg when the positive voltage is applied (in the forward bias state) in comparison to a release amount (e.g., 31 μg) in a passive state in which no voltage is applied. In addition, it can be found that the release amount of the ionic material decreases to 10 μg when the negative voltage is applied (in the reverse bias state) in comparison to the release amount (e.g., 31 μg) in the passive state in which no voltage is applied.

In other words, it can be found based on the graph 830 that the largest amount of ionic material is released when the forward bias voltage is applied to the bipolar membrane, and that a smallest amount of ionic material is released when the reverse bias voltage is applied to the bipolar membrane.

The delivery device may adjust the release amount and/or a release rate of the ionic material passing through the bipolar membrane by adjusting a direction in which an electric field is formed and an intensity of the electric field.

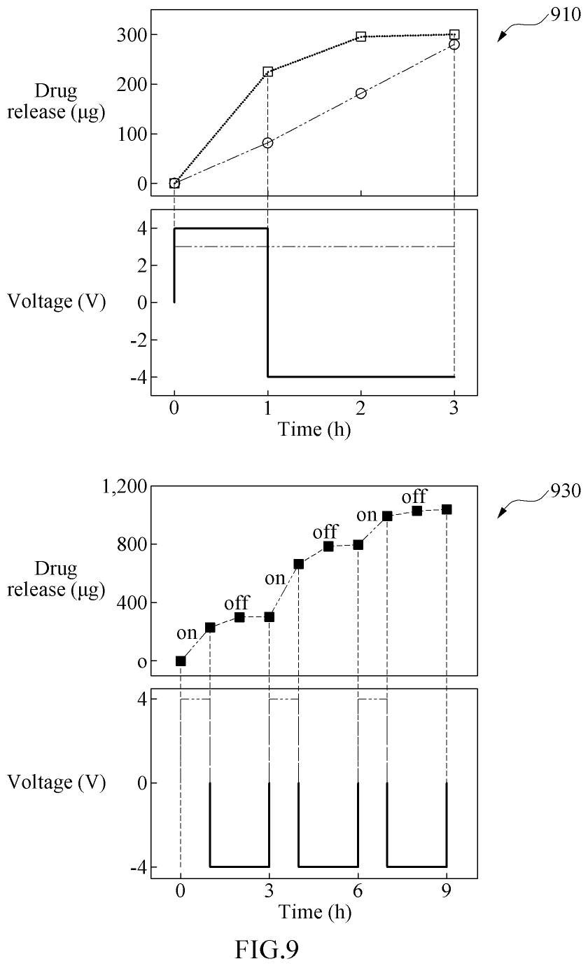

FIG. 9 illustrates examples of a release amount of an ionic material according to a bias voltage.

Referring to FIG. 9, a graph 910 may represent a result obtained by measuring the release amount of the ionic material according to the bias voltage. In the graph 910, it can be found that a release amount of the ionic material measured when a forward bias of 4 volts (V) is applied for one hour and a reverse bias of −4V is then applied for two hours, as indicated by a solid line, and a release amount of the ionic material measured when a forward bias of 3 V is applied for three hours, as indicated by a dash-double dotted line, are the same as 300 μg.

In addition, a graph 930 may represent a result obtained by performing a time-dependent adjustment of a bias voltage and repeated release experiments. For example, it can be confirmed based on the graph 930 that a predetermined amount of drug is released when a forward bias indicated by a dash-double dotted line and a reverse bias indicated by a solid line are repeatedly applied with the same voltage strength (e.g., ±4V).

In an example, a delivery device may diversify an ionic material release strategy by adjusting a bias intensity and/or a bias direction based on measurement results of the graphs 910 and 930.

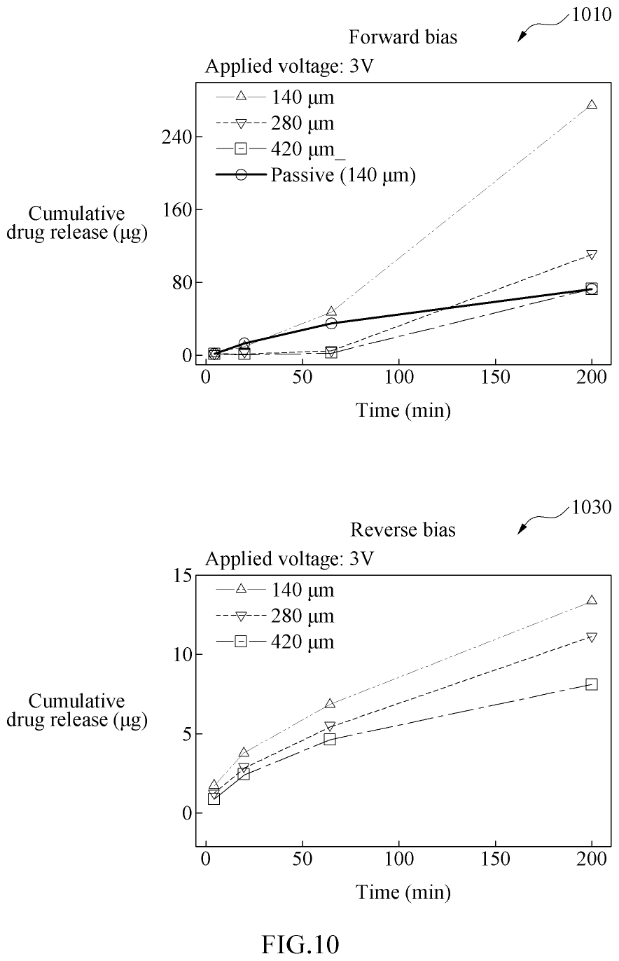

FIG. 10 illustrates an example of a change in a release amount of an ionic material according to a thickness of a bipolar membrane for each bias voltage.

Referring to FIG. 10, a graph 1010 may represent a release amount of an ionic material according to different thicknesses (e.g., 140 μm, 280 μm, and 420 μm) of a bipolar membrane in a forward bias. In addition, a graph 1030 may represent a release amount of an ionic material according to different thicknesses (e.g., 140 μm, 280 μm, and 420 μm) of the bipolar membrane in a reverse bias.

Referring to the graphs 1010 and 1030, it can be confirmed that as the thickness of the bipolar membrane increases in the forward bias and the reverse bias, finely control of a release of the ionic material is possible. In an example, a bipolar membrane having an appropriate thickness may be used in a delivery device based on experimental results of FIG. 10.

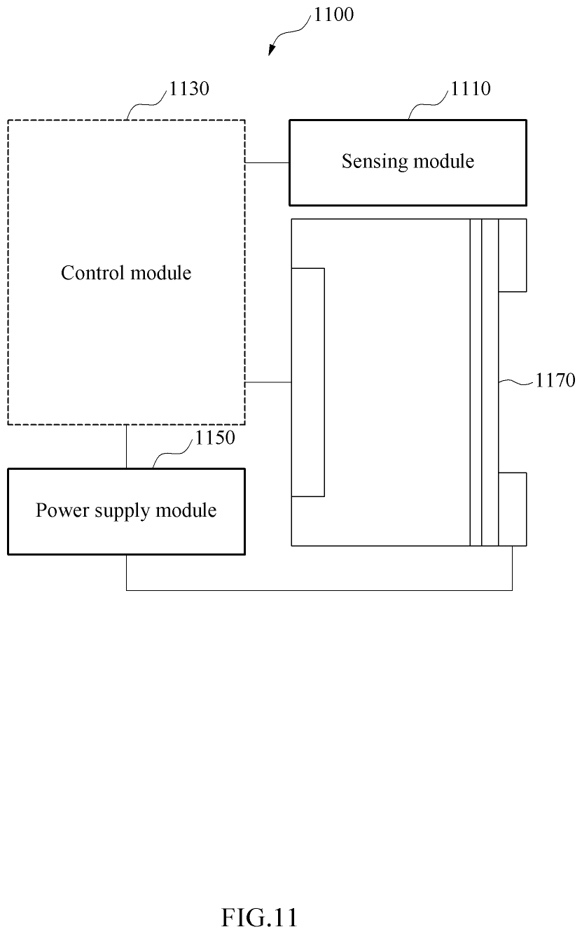

FIG. 11 is a block diagram of another example of a delivery device. Referring to FIG. 11, a delivery device 1100 may include a sensing module 1110, a control module 1130, a power supply module 1150, and an ionic material carrier 1170.

The delivery device 1100 of FIG. 11 may be a closed-loop delivery device configured by adding the sensing module 1110 to the delivery device 100 of FIG. 1. The control module 1130, the power supply module 1150, and the ionic material carrier 1170 of the delivery device 1100 may respectively correspond to the control module 130, the power supply module 150, and the ionic material carrier 110 of the delivery device 100 of FIG. 1, and a configuration and operation different from those of the delivery device 100 will be mainly described below.

The sensing module 1110 may detect any one or any combination of a state of a human body into which the delivery device 1100 is inserted, and an amount and a concentration of a feature material, including an ionic material in the human body.

The control module 1130 may determine any one or any combination of whether to release the ionic material and a release time, a release period, a release rate, and a release amount of the ionic material, based on a detection result of the sensing module 1110. The control module 1130 may control either one or both of a direction in which an electric field is formed and an intensity of the electric field based on a determination of whether to release the ionic material and the release time, the release period, the release rate, and the release amount of the ionic material.

For example, the delivery device 1100 may move the ionic material into a body using the electric field applied to a bipolar membrane of the ionic material carrier 1170, instead of allowing drug to flow into the body through an irreversible operation such as electrolysis of a metal thin film, thereby easily controlling a concentration and/or a release rate of the drug. In addition, the delivery device 1100 may detect an amount and a concentration of a feature material including an ionic material in a human body in real time, and may arbitrarily adjust a release amount of the ionic material according to a user's condition, thereby providing an appropriate dose of drug according to patient's individual circumstances.

FIG. 12 is a block diagram of another example of a delivery device. Referring to FIG. 12, a delivery device 1200 may include a wireless power reception module 1210, a communication module 1220, a control module 1230, and an ionic material carrier 1240.

The control module 1230 and the ionic material carrier 1240 of the delivery device 1200 may respectively correspond to the control module 130 and the ionic material carrier 110 of the delivery device 100 shown in FIG. 1, and a configuration and operation different from those of the delivery device 100 will be mainly described below.

Unlike the delivery device 100 of FIG. 1, the delivery device 1200 of FIG. 12 may be a delivery device configured to perform charging and/or a release of the ionic material based on a control signal from an external device 1250. Unlike the external device 1250 located outside a body, the delivery device 1200 may be inserted into the body.

The external device 1250 may be, for example, one of a user terminal such as a smartphone, a wearable device, other medical devices, and a cloud server, but is not limited thereto. The external device 1250 may include a communication module 1251, and a wireless power transmission module 1253. The communication module 1251 may transmit a control signal generated by the external device 1250 to the delivery device 1200. The wireless power transmission module 1253 may wirelessly supply power to the wireless power reception module 1210.

The wireless power reception module 1210 may wirelessly receive the power from the external device 1250 and use the power to operate the delivery device 1200.

The communication module 1220 may receive a control signal from the external device 1250.

The control module 1230 may determine whether to release the ionic material through the ionic material carrier 1240, and an amount of drug to be released from the ionic material carrier 1240, based on the control signal received via the communication module 1220. The control signal may correspond to, for example, a signal for controlling any one or any combination of whether to release the ionic material through the ionic material carrier 1240, and a release time, a release rate, and a release amount of the ionic material, but is not limited thereto.

The control module 1230 may release the ionic material into the body by applying a desired voltage to the ionic material carrier 1240 according to a determination based on the control signal.

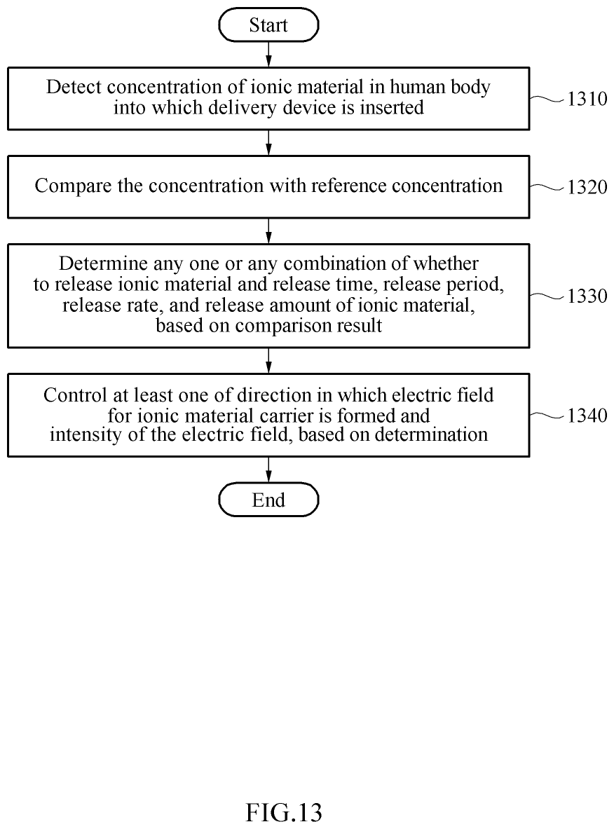

FIG. 13 is a flowchart illustrating an example of a method of controlling a delivery device. In the following examples, operations may be performed sequentially, but not necessarily performed sequentially. For example, the order of the operations may be changed and at least two of the operations may be performed in parallel.

Referring to FIG. 13, the delivery device may control either one or both of a release amount and a release period of an ionic material through operations 1310 to 1340. The delivery device may be, for example, one of the delivery device 100 of FIG. 1, the delivery device 300 of FIGS. 3A and 3B, and the delivery device 1100 of FIG. 11, but is not limited thereto.

In operation 1310, the delivery device may detect a concentration of the ionic material in a human body into which the delivery device, including an ionic material carrier, is inserted.

In operation 1320, the delivery device may compare the concentration detected in operation 1310 with a reference concentration. For example, the reference concentration may be individually determined for each user (or patient) into which the delivery device is inserted, or may be determined for each type of ionic materials released into a human body, however, the examples are not limited thereto.

In operation 1330, the delivery device may determine any one or any combination of whether to release the ionic material and a release time, a release period, a release rate, and a release amount of the ionic material, based on a comparison result obtained in operation 1320.

In an example, if the concentration detected in operation 1310 is less than the reference concentration, the delivery device may determine to release the ionic material in operation 1330, and determine any one or any combination of the release time, the release rate, and the release amount such that the ionic material may be released in proportion to a percentage corresponding to a difference between the concentration detected in operation 1310 and the reference concentration. In this example, if the percentage is 50% or greater, the delivery device may increase a value of any one or any combination of the release time, the release rate, and the release amount by 50% or greater.

In another example, if the concentration detected in operation 1310 is greater than or equal to the reference concentration, the delivery device may determine to block a release of the ionic material in operation 1330.

In operation 1340, the delivery device may control either one or both of a direction in which an electric field for the ionic material carrier is formed, and an intensity of the electric field, based on a determination of operation 1330.

In an example, if it is determined to release the ionic material in operation 1330 because the concentration detected in operation 1310 is less than the reference concentration, the delivery device may control the direction in which the electric field is formed such that a forward bias may be formed with respect to the ionic material carrier. In this example, in operation 1340, the delivery device may control either one or both of the intensity of the electric field supplied to the ionic material carrier and/or a power supply time to match any one or any combination of any two or more of the release time, the release rate, and the release amount determined in operation 1330.

In another example, if it is determined to block the release of the ionic material in operation 1330, the delivery device may control the direction in which the electric field is formed such that a reverse bias may be formed with respect to the ionic material carrier in operation 1340.

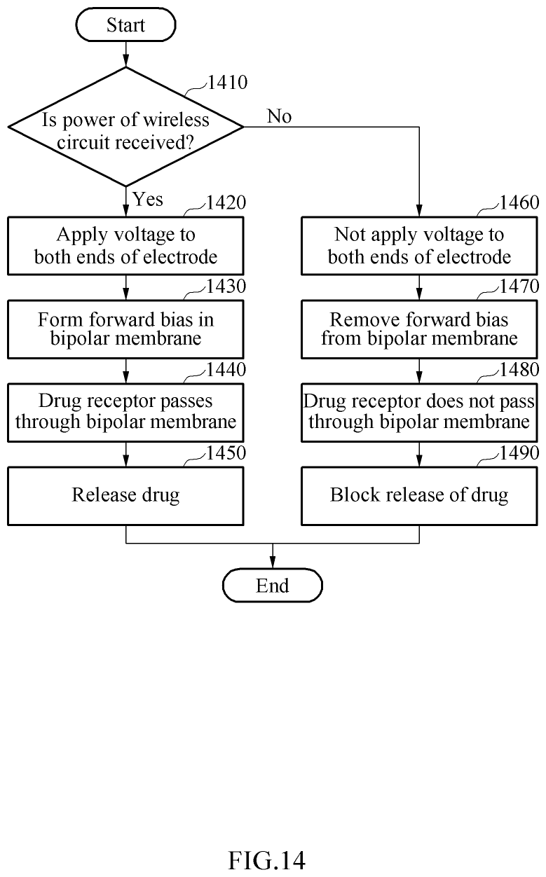

FIG. 14 is a flowchart illustrating another example of a method of controlling a delivery device. In the following examples, operations may be performed sequentially, but not necessarily performed sequentially. For example, the order of the operations may be changed and at least two of the operations may be performed in parallel.

FIG. 14 illustrates a process in which drug is delivered into a human body by wireless electrophoresis in a delivery device. The delivery device may be, for example, the delivery device 1200 of FIG. 12, but is not limited thereto.

In operation 1410, the delivery device may determine whether the power of a wireless circuit is received. In an example, if it is determined in operation 1410 that the power of the wireless circuit is received, the delivery device may apply a voltage to both ends of an electrode of an ionic material carrier in operation 1420 and may form a forward bias in a bipolar membrane in operation 1430. A drug receptor may pass through the bipolar membrane by the forward bias in operation 1440, and accordingly the delivery device may release drug in operation 1450.

In another example, if it is determined in operation 1410 that the power of the wireless circuit is not received, the delivery device may not apply a voltage to the ends of the electrode of the ionic material carrier in operation 1460 and remove a forward bias from the bipolar membrane in operation 1470. If the drug receptor does not pass through the bipolar membrane in operation 1480 by removing the forward bias, the delivery device may block a release of drug in operation 1490.

The delivery device, ionic material carrier, control module, ionic material carrier 110, control module 130, delivery device 100, 300, 1100, 1200 in FIGS. 1-14 that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-14 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A device for delivering an ionic material, the device comprising:
a storage module comprising a reservoir configured to store the ionic material;
a bipolar membrane configured to pass the ionic material in a single direction based on an ionic current;
electrodes, disposed on a lower end of the reservoir and an upper end of the bipolar membrane, respectively, configured to form an electric field generating the ionic current; and
a control module configured to control either one or both of a release amount and a release period of the ionic material passing through the bipolar membrane by adjusting a direction and an intensity of the electric field.

2. The device of claim 1, wherein the control module is further configured to:
control the ionic material to be released through the bipolar membrane by generating the ionic current based on a forward bias voltage provided by applying a cathode voltage to a region adjacent to a cationic polymer of the bipolar membrane and applying an anode voltage to a region adjacent to an anionic polymer of the bipolar membrane through the electrodes.

3. The device of claim 1, wherein the control module is further configured to:
control the ionic material not to pass through the bipolar membrane by blocking generation of the ionic current by forming a depletion layer in a bonding portion of the bipolar membrane based on a reverse bias voltage provided by applying an anode voltage to a region adjacent to a cationic polymer of the bipolar membrane and applying a cathode voltage to a region adjacent to an anionic polymer of the bipolar membrane through the electrodes.

4. The device of claim 1, further comprising:
a sensing module configured to detect either one or both of an amount and a concentration of the ionic material in a human body into which the device is inserted.

5. The device of claim 4, wherein the control module is further configured to:
determine any one or any combination of any two or more of whether to release the ionic material, the release amount, and the release period of the ionic material according to a detection result of the sensing module; and
adjust the direction and the intensity of the electric field based on a result of the determine.

6. The device of claim 1, wherein
the bipolar membrane comprises an anionic polymer having a first permeability to ions having same charges and a cationic polymer having a second permeability to ions having opposite charges, and
the second permeability is greater than the first permeability.

7. The device of claim 6, wherein
the anionic polymer comprises one of sulfonated polyphenylene oxide, sulfonated polyethersulfone, sulfonated polyether ether ketone, sulfonated polystyrene, phosphorylated polyphenylene oxide, phosphorylated polysulfone, and carboxylated polyethylene, and
the cationic polymer comprises one of quaternized polyphenylene oxide, quaternized polysulfone, imidazolated polyphenylene oxide, quaternized polyether ether ketone, amidated polyphenylene oxide, amidated polysulfone, and amidated polyether ether ketone.

8. The device of claim 1, further comprising:
a drug receptor accommodated in the reservoir,
the drug receptor comprising the ionic material and any one or any combination of any two or more of phosphate-buffered saline, an aqueous sodium chloride solution, an agarose gel, an alginate gel, and a chitosan gel.

9. The device of claim 1, wherein
the storage module is further configured to store a cell generating and secreting a therapeutic factor, and
the bipolar membrane is further configured to store or release the therapeutic factor based on the ionic current.

10. The device of claim 1, wherein the storage module comprises one of polybutylene adipate terephthalate (PBAT), polyurethane, polyethylene, polysulfone, polydimethylsiloxane, and polymethyl methacrylate.

11. The device of claim 1, wherein the electrodes comprise any one or any combination of any two or more of platinum, gold, stainless steel, silver, silver chloride, carbon, and oxides thereof.

12. The device of claim 1, further comprising:
a communication module configured to receive a control signal based on the release amount and the release period of the ionic material.

13. The device of claim 1, further comprising:
a wireless power reception module configured to wirelessly receive power inducing the electric field.

14. The device of claim 1, further comprising:
a power supply module configured to supply power inducing the electric field.

15. A method of delivering an ionic material in a device, the method comprising:
detecting a concentration of the ionic material in a human body into which the device is inserted;
comparing the concentration with a reference concentration;
determining any one or any combination of any two or more of whether to release the ionic material and a release time, a release period, a release rate, and a release amount of the ionic material, based on a result of the comparing; and
controlling either one or both of a direction and an intensity of an electric field of an ionic material carrier of the device based on the determining.

16. The method of claim 15, wherein the determining comprises:
determining to release the ionic material in response to the concentration being less than the reference concentration; and
determining any one or any combination of any two or more of the release time, the release rate, and the release amount to release the ionic material in proportion to a difference between the concentration and the reference concentration.

17. The method of claim 16, wherein the controlling comprises controlling the direction of the electric field to form a forward bias with respect to the ionic material carrier in response to a determination to release the ionic material.

18. The method of claim 15, wherein the determining comprises determining to block a release of the ionic material in response to the concentration being greater than or equal to the reference concentration.

19. The method of claim 18, wherein the controlling comprises controlling the direction of the electric field to form a reverse bias with respect to the ionic material carrier in response to a determination to block the release of the ionic material.

20. A non-transitory computer-readable storage medium storing instructions that, when executed by one or more processors, configure the one or more processors to perform the method of claim 15.
